(19) United States
(12) Patent Application Publication
(10) Pub. No.: US 2018/0360406 A1
(43) Pub. Date: Dec. 20, 2018

(54) Image Processing Device and Image Processing Method

(71) Applicant: The University of Tokyo, Chuo-ku, Tokyo (JP)

(72) Inventors: Akihiro HAGA, Tokyo (JP); Taiki MAGOME, Tokyo (JP); Keiichi NAKAGAWA, Tokyo (JP)

(21) Appl. No.: 16/061,600

(22) PCT Filed: Dec. 14, 2016
(86) PCT No.: PCT/JP2016/087199

§ 371 Date: Jul. 26, 2018

(30) Foreign Application Priority Data
Dec. 17, 2015 (JP) 2015-246604

(51) Int. Cl. A61B 6/00; A61B 6/03; A61N 5/10; G06T 11/00

(52) U.S. Cl. CPC A61B 6/5205; A61B 6/5235; A61B 6/032; G06T 2211/424; G06T 11/006; A61N 2005/1061; G06T 2211/421; A61N 5/1049

(57) ABSTRACT

Provided are an image processing apparatus and an image processing method that can suitably broaden a field of view. The image processing apparatus includes: first input means for receiving input of sinogram information acquired by projecting radiation onto an object; means for configuring a first tomographic image of the object from the sinogram information; second input means for receiving input of a prior tomographic image obtained by imaging the object before the sinogram information; conversion means for converting a pixel value of the prior tomographic image on the basis of a pixel value of the first tomographic image; and means for generating a second tomographic image from the sinogram information with use of the prior tomographic image, the pixel value of which has been converted.

### TECHNICAL FIELD

A plurality of aspects according to the present invention relate to an image processing apparatus and an image processing method for processing a tomographic image of a human body, for example.

### BACKGROUND ART

A computed tomography (hereinafter also referred to as “CT”) apparatus that acquires a tomographic image of an object such as a human body by irradiating the object with radiation and detecting the transmitted radiation is widely used. Because the inside of the human body such as visceral organs can be photographed, the computed tomography apparatus is widely used in fields of diagnosis and the like.

Image pickup apparatuses such as the CT apparatus as above have a field of view in which the image of the object can be suitably restored. When the object is out of the field of view, the tomographic image of the object cannot be suitably configured because sufficient information cannot be acquired, for example. Therefore, Patent Document 1 alleviates the incompleteness of the area outside the field of view by using data adjusted with use of a morphological filter, for example, together with the original imaging data.

### CITATION LIST

### Patent Document

Patent Document 1: U.S. Patent Application Publication No. 2013/0301894 (Specification)

### SUMMARY

### Technical Problem

However, the method disclosed in Patent Document 1 only slightly alleviates the incompleteness of the area outside the field of view and it is a stretch to say that the field of view is sufficiently broadened. In particular, the field of view is generally narrow when the tomographic image is photographed with a position collation CT apparatus and the like accompanying a radiation therapy apparatus instead of a diagnostic CT apparatus, and hence an image processing method that can sufficiently broaden the field of view is desired.

A plurality of aspects of the present invention have been made in view of the abovementioned problem, and an object thereof is to provide an image processing apparatus and an image processing method that can suitably broaden a field of view.

### Solution to Problem

An information processing apparatus according to one aspect of the present invention includes: first input means for receiving input of sinogram information acquired by projecting radiation onto an object; means for configuring a first tomographic image of the object from the sinogram information; second input means for receiving input of a prior tomographic image obtained by imaging the object before the sinogram information; conversion means for converting a pixel value of the prior tomographic image on the basis of a pixel value of the first tomographic image; and means for generating a second tomographic image from the sinogram information with use of the prior tomographic image, the pixel value of which has been converted.

An information processing method according to one aspect of the present invention includes performing, by an information processing apparatus, the step of receiving input of sinogram information acquired by projecting radiation onto an object, the step of configuring a first tomographic image of the object from the sinogram information, the step of receiving input of a prior tomographic image obtained by imaging the object before the sinogram information, the step of converting a pixel value of the prior tomographic image on the basis of a pixel value of the first tomographic image, and the step of generating a second tomographic image from the sinogram information with use of the prior tomographic image, the pixel value of which has been converted.

In the present invention, the expressions of “unit”, “means”, “apparatus”, and “system” not only mean physical means, but also include a case where the functions of the “unit”, the “means”, the “apparatus”, and the “system” are realized by software. A function of one “unit”, “means”, “apparatus”, or “system” may be realized by two or more physical means or apparatuses, or functions of two or more “units”, “means”, “apparatuses”, and “systems” may be realized by one physical means or apparatus.

### BRIEF DESCRIPTION OF DRAWINGS

FIG. 1 is a block diagram illustrating a function configuration of an image processing apparatus according to an embodiment.

FIG. 2 is a flowchart illustrating a flow of processing of the image processing apparatus illustrated in FIG. 1.

FIG. 3 is a specific example of images processed by the image processing apparatus illustrated in FIG. 1.

FIG. 4 is a block diagram illustrating a specific example of a hardware configuration capable of implementing the image processing apparatus illustrated in FIG. 1.

### DESCRIPTION OF EMBODIMENTS

An embodiment of the present invention is described below with reference to the drawings. However, the embodiment described below is only an example and is not intended to exclude various modifications or application of technology that are not explicitly noted below. That is, the present invention can be embodied with various modifications without departing from the gist thereof. In the description of the drawings below, the same or similar parts are denoted by the same or similar symbols. The drawings are schematic and do not necessarily match with the actual sizes, ratios, and the like. The drawings may include parts having size relationships or ratios that differ among the drawings.

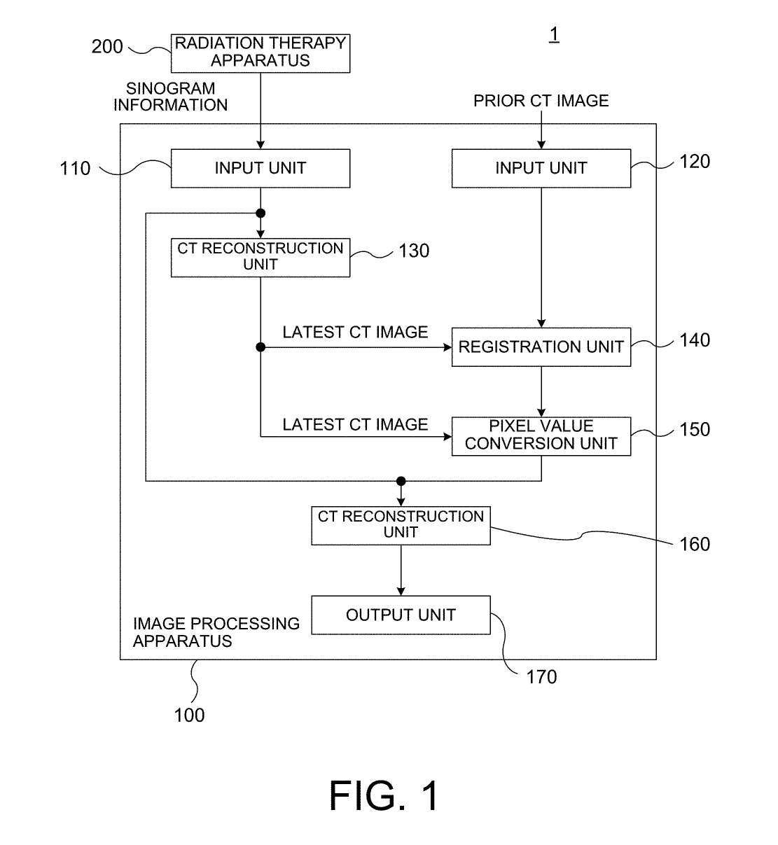

FIG. 1 to FIG. 4 are diagrams for describing the embodiment. The embodiment is described along a flow below with reference to the drawings below. First, an overview of an image processing apparatus according to the embodiment is described in “1”. Then, a function configuration of the image processing apparatus is described in “2” and a flow of processing of the image processing apparatus is described in “3”. In “4”, an example of a result obtained by the processing with use of the image processing apparatus is described. In “5”, a specific example of a hardware configuration capable of realizing the image processing apparatus is described. Lastly, an effect and the like according to the embodiment are described from “6” and thereafter.

(1. Overview)

A computed tomography (hereinafter also referred to as “CT”) apparatus is widely used when a tomographic image of an object such as a human body is generated. A typical CT apparatus is configured so that, in a ring-like gantry, a radiator that emits radiation toward the direction of the center of a ring, and a detector that detects the emitted radiation can circumferentially travel. A couch on which the object is laid moves to a place near the center of the ring. As a result, the object is rotationally irradiated with the radiation. The radiation that has passed through the object is detected by the abovementioned detector, and sinogram information in which projection images for each angle are arranged in a longitudinal direction is firstly generated. The tomographic image of the object can be acquired by performing CT reconstruction on the sinogram information.

When it is considered that the radiation therapy is performed for cancer therapy and the like, a doctor photographs the tomographic image of a patient by the CT apparatus after fixing the patient on the couch of the CT apparatus. The doctor identifies the affected part of the cancer and the like by observing and diagnosing the tomographic image.

Then, the affected part of the patient is irradiated with radiation while the patient is fixed on the couch of the radiation therapy apparatus when the radiation therapy is performed on the patient. In general, the radiation used in this therapy has a narrower irradiation width and higher intensity than the radiation used by a diagnostic CT apparatus. Therefore, it is important to suitably register the patient on the couch before the irradiation of the radiation for the therapy in order to securely irradiate the affected part with the radiation for the therapy applied by the radiation therapy apparatus while preventing the radiation for the therapy from being applied on other parts of the patient and to adjust the radiation to have a suitable intensity. More specifically, there is a need to register and fix the patent on the couch so that the affected part of the patient is placed at the position that can be irradiated with the radiation for the therapy and the posture of the patient is about the same as that when the tomographic image for diagnostic use is photographed. Therefore, the latest radiation therapy apparatuses generally have a CT function by a position collation CT apparatus for photographing the tomographic image used for the registration and the like.

However, the radiation therapy apparatus is only for irradiating the affected part with radiation for therapy, and hence the size of the position collation CT apparatus for photographing a tomographic image that is not directly related with the radiation therapy cannot be sufficiently ensured. As a result, it is difficult for the position collation CT apparatus included in the radiation therapy apparatus to ensure a wide field of view for suitably photographing the tomographic image. Therefore, the position collation CT apparatus of the radiation therapy apparatus generally has a narrower field of view than the diagnostic CT apparatus. As described above, there is a need to place the affected part in a position easily irradiated with the radiation for therapy such as the center of the couch, and hence it is often difficult to fit the entire tomogram of the body of the patient in the field of view. Meanwhile, in the radiation therapy, there is a need to adjust the radiation amount to be applied in accordance with the distance from the surface of the patient that is the object to the affected part, and hence it is desired that the entire tomogram of the patient including areas other than the affected part can be imaged.

Therefore, the image processing apparatus according to this embodiment broadens the field of view by supplying missing information with use of a prior tomographic image photographed in advance for diagnosis, for example.

(2. Function Configuration of Image Processing Apparatus)

A function configuration of an image processing system 1 according to this embodiment is described below with reference to FIG. 1. FIG. 1 is a functional block diagram illustrating a specific example of the function configuration of the image processing system 1. The image processing system 1 includes an image processing apparatus 100 and a radiation therapy apparatus 200.

In the example of FIG. 1, the image processing apparatus 100 and the radiation therapy apparatus 200 are described as physically different apparatuses but are not limited thereto and may be implemented as the radiation therapy apparatus 200 having the function of the image processing apparatus 100, for example. Alternatively, the function of the image processing apparatus 100 may be separately realized in a plurality of information processing apparatuses.

The radiation therapy apparatus 200 is an apparatus for treating cancer and the like by irradiating the affected part of the patient by radiation. In this embodiment, the radiation therapy apparatus 200 has a CT function for photographing the tomographic image in order to register the patient before the therapy, for example. The radiation therapy apparatus 200 outputs the sinogram information acquired by the CT function to the image processing apparatus 100.

The image processing apparatus 100 receives the input of the sinogram information from the radiation therapy apparatus 200, receives the input of the prior tomographic image (also referred to as a “prior CT image”) obtained by photographing the same patient in advance, and generates the tomographic image of the patient on the basis of the input. The image processing apparatus 100 according to this embodiment includes input units 110 and 120, a CT reconstruction unit 130, a registration unit 140, a pixel value conversion unit 150, a CT reconstruction unit 160, and an output unit 170.

The input unit 110 of the image processing apparatus 100 receives the input of the sinogram information output from the radiation therapy apparatus 200. The input unit 120 receives the input of the prior CT image photographed in advance by the diagnostic CT apparatus, for example. The prior CT image input from the input unit 120 does not necessarily need to be photographed by the diagnostic CT apparatus. For example, the tomographic image of the same patient generated by the image processing apparatus 100 before may be used as the prior CT image.

The CT reconstruction unit 130 generates the latest CT image showing the current tomogram of the patient by performing CT reconstruction of the sinogram information input from the input unit 120. A filtered-back projection (FBP) method is used for the CT reconstruction, for example.

The registration unit 140 registers the prior CT image input from the input unit 120 with the latest CT image generated by the CT reconstruction unit 130. There are various methods for the registration. For example, a difference in pixel values between the latest CT image and the prior CT image may be calculated for all pixels by using the latest CT image as a reference, and the position of the prior CT image at which the total amount of differences in pixel values is small may be obtained.

The pixel value conversion unit 150 causes the pixel values of the prior CT image to match the level of the pixel values of the latest CT image by linearly or non-linearly converting the pixel values of the prior CT image on the basis of the pixel values of the latest CT image. The diagnostic CT apparatus applies relatively low radiation that is a kilo-voltage level, while the radiation therapy apparatus 200 sometimes uses intense radiation that is a mega-voltage level for therapy. When the radiation level applied to the object changes, the radiation level detected by the detector also changes, and hence the pixel value level of the tomographic image generated on the basis of the detected radiation also changes. Therefore, there is a need to cause the pixel value levels of both images to be even by the pixel value conversion unit 150.

There are various methods for obtaining the conversion expression to be applied in the pixel value conversion unit 150. For example, a combination of a tomographic image photographed by the radiation therapy apparatus 200 and a tomographic image photographed by the CT apparatus that has photographed the prior CT image may be prepared for a plurality of samples, and a linear conversion expression in which the pixel value levels of both tomographic images are approximated may be obtained.

The CT reconstruction unit 160 performs CT reconstruction by methods such as an iterative reconstruction (IR, hereinafter also referred to as “IR method”) or the filtered-back projection (FBP) with use of the prior CT image, which is registered and the pixel value level of which has been adjusted, and the sinogram information input from the input unit 110. When the IR method is used, for example, an object function of the IR method is defined by Expression (1).

$$\begin{matrix}[ Math . 1 ] & \\ \hat{I} = {arg max}_{I} \{ ln ( p ( n \vert I ) ) + ln ( R ( I ) ) \} & ( 1 )\end{matrix}$$

### in Expression (1),

$$\begin{matrix}[ Math . 2 ] & \\ \hat{I} & \end{matrix}$$

represents an image after the CT reconstruction to be calculated. In addition, p(n|I) represents a conditional probability that a number of n photons are observed when a reconstruction image I is provided. For example, p(n|I) can be defined by Expression (2) as a Poisson distribution. However, p(n|I) can also be defined by other expressions.

$$\begin{matrix}[ Math . 3 ] & \\ p ( n \vert I ) = {\prod }_{i = 1}^{M} \frac{{( {n}_{0} {e}^{- {\sum }_{j} {a}_{ij} {I}^{*} j} )}^{{n}_{i}}}{{n}_{i} !} {e}^{- {n}_{0} {e}^{- {\sum }_{j} {a}_{ij} {I}^{*} j}} & ( 2 )\end{matrix}$$

In Expression (2), n₀ represents an initial number of photons emitted to the i-th detector cell. In addition, aᵢⱼ represents the length of the beamlet which passed the j-th voxel, I*ⱼ represents an expected value of a linear attenuation coefficient of the j-th voxel, nᵢ represents the number of photons observed in the i-th detector cell, and M represents the product of the number of detector cells and the number of projections used for reconstruction of slices. Among the values, nᵢ is acquired from the sinogram information.

For example, In(R(I)) in Expression (1) is calculated on the basis of Expression (3).

$$\begin{matrix}[ Math . 4 ] & \\ ln ( R ( I ) ) = - {w}_{TV} TV ( I ) - {w}_{p} {‖ I - Ip ‖}_{1} & ( 3 )\end{matrix}$$

In Expression (3), w_{TV} and wₚ represent constants, TV(I) represents a penalty term relating to the total variation, and Ip represents the prior CT image. For example, TV(I) in Expression (3) is calculated by Expression (4).

$$\begin{matrix}[ Math . 5 ] & \\ \begin{matrix}TV ( I ) = {\int }_{Ω} \vert ∇ I \vert dxdy \\ \approx {\sum }_{m , n} \sqrt{{( {I}_{m , n} - {I}_{m + 1 , n} )}^{2} + {( {I}_{m , n} - {I}_{m , n + 1} )}^{2}}\end{matrix} & ( 4 )\end{matrix}$$

In Expression (4), m and n represent voxel numbers in x and y directions in the reconstruction image I.

As described above, the CT reconstruction unit 160 may perform the CT reconstruction by the FBP method instead of the IR method. In that case, the CT reconstruction area can be expanded by generating the sinogram information from the prior CT image, which is registered and the pixel value level of which has been adjusted, through computation, and by supplying the missing area in the original sinogram information with the sinogram information generated by the computation. When the FBP method is used, the computation speed can be enhanced as compared to the IR method.

The output unit 170 outputs the CT image reconstructed by the CT reconstruction unit 160 to a display apparatus or a storage apparatus, for example.

(3. Flow of Processing)

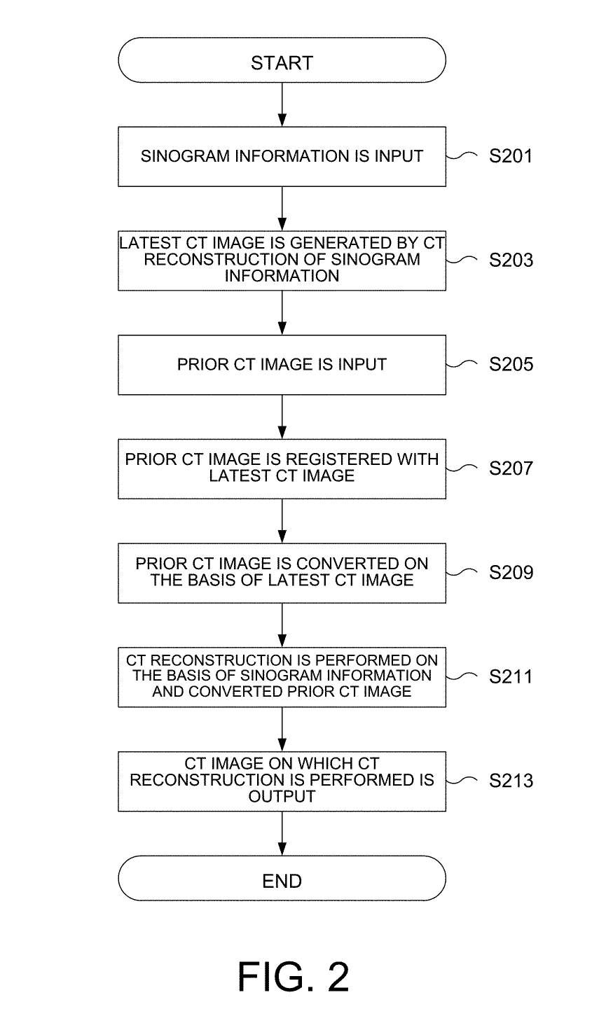

A flow of the processing of the image processing apparatus 100 is described below with reference to FIG. 2. FIG. 2 is a flowchart illustrating the flow of the processing of the image processing apparatus 100 according to this embodiment.

The processing steps described below can be executed with the order thereof freely changed or in parallel with each other as long as there is no inconsistency in the processing content. Other steps may be added between the processing steps. Steps described as one step for convenience can be executed as a plurality of separate steps and a step described as a plurality of separate steps for convenience can be executed as one step.

First, the input unit 110 receives the input of the sinogram information from the radiation therapy apparatus 200 (S201), and the CT reconstruction unit 130 reconstructs the latest CT image from the input sinogram information with use of an FBP algorithm, for example. The sinogram information input from the radiation therapy apparatus 200 is photographed by the radiation therapy apparatus 200 for the registration of the patient before the radiation therapy, for example.

The input unit 120 receives the input of the prior CT image from the storage apparatus or an external information processing apparatus, for example (S205). The registration unit 140 registers the prior CT image input from the input unit 120 with the latest CT image generated by the CT reconstruction unit 130 (S207). The pixel value conversion unit 150 adjusts the pixel values of the prior CT image by converting the pixel values of the prior CT image on the basis of the pixel values of the latest CT image (S209).

When the registration and the adjustment of the pixel values of the prior CT image are finished, the CT reconstruction unit 160 reconstructs the CT image of the sinogram information input from the input unit 110 with use of the prior CT image on which the abovementioned processing has been performed (S211). The reconstructed CT image is output to the display apparatus or the storage apparatus by the output unit 170 (S213).

(4. Specific Example of Reconstructed CT Image)

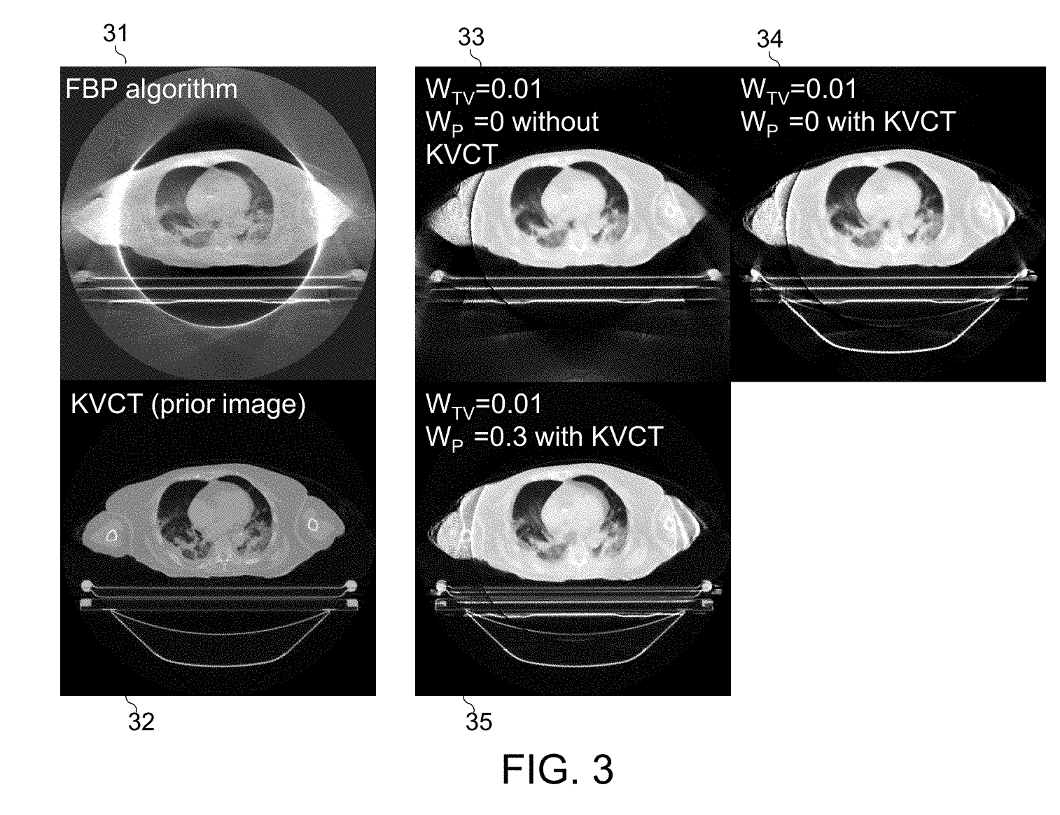

FIG. 3 illustrates a specific example of CT images generated by the image processing apparatus 100 according to this embodiment. Images 31 and 32 shown on the left side in FIG. 3 are the latest CT image and the prior CT image. All the images in FIG. 3 are obtained by imaging the chest of the patient that is the object. In FIG. 3, the image 31 that is the latest CT image is generated with use of the FBP algorithm from the sinogram information generated by irradiating the patient that is the object with radiation from the periphery for 216 degrees. The image 32 that is the prior CT image is photographed by kVCT (kilovoltage computed tomography), that is, the CT apparatus.

In the image 31, the substantially circular area in the center is the field of view and a chest tomogram of the patient is suitably restored. However, the entire circumferential area in the periphery including the arms and the like of the patient comes out whitish, and the tomogram of the arms of the patient is not reproduced well in the image 31 as compared to the image 32.

Images 33 to 35 on the right side in FIG. 3 are CT images reconstructed by the IR method with use of Expressions (1) to (4) as above. In particular, in the image 35 generated by setting 0.01 and 0.3 in the parameters w_{TV} and wₚ, it can be seen that the arms and the like that are not sufficiently reproduced in the image 31 are reproduced with use of information of the image 32 that is the prior CT image. That is, the field of view has become broader.

(5. Specific Example of Hardware Configuration)

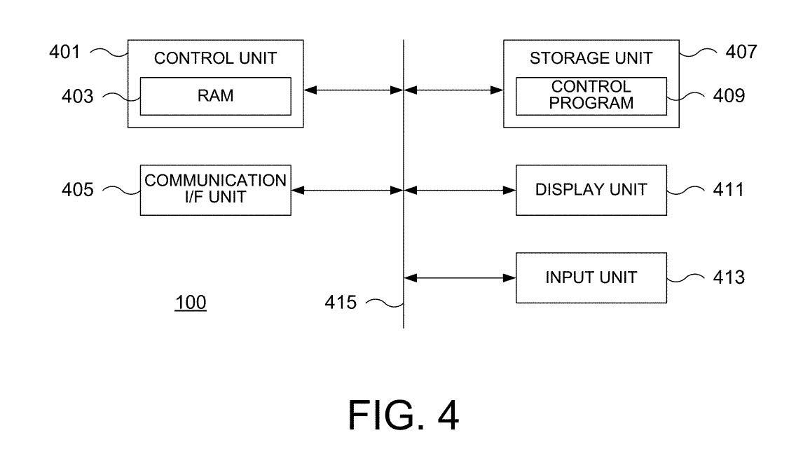

A specific example of a hardware configuration of the image processing apparatus 100 is described below with reference to FIG. 4. As illustrated in FIG. 4, the image processing apparatus 100 includes a control unit 401, a communication interface (I/F) unit 405, a storage unit 407, a display unit 411, and an input unit 413, and the units are connected to each other via a bus line 415.

The control unit 401 includes a CPU (Central Processing Unit, not shown), a ROM (Read Only Memory, not shown), a RAM (Random Access Memory) 403, and the like. The control unit 401 is configured to be able to execute the abovementioned image processing in addition to functioning as a typical computer by executing a control program 409 stored in the storage unit 407. For example, the input unit 110, the input unit 120, the CT reconstruction unit 130, the registration unit 140, the pixel value conversion unit 150, the CT reconstruction unit 160, and the output unit 170 described with reference to FIG. 1 can be realized as the control program 409 that is temporarily stored in the RAM 403 and operates on the CPU.

The RAM 403 temporarily holds a part or all of the sinogram information, the prior CT image, the latest CT image, and the like other than codes included in the control program 409. The RAM 403 is also used as a working area when the CPU executes various processing.

The communication I/F unit 405 is a device for communicating data in a wired or wireless manner with the radiation therapy apparatus 200, the storage apparatus storing the prior CT image therein, or other information processing apparatuses, for example. For example, the communication I/F unit 405 can be used when the input units 110 and 120 receive the input of the sinogram information or the prior CT image.

The storage unit 407 is a nonvolatile storage medium such as an HDD (Hard Disk Drive) or a flash memory. The storage unit 407 stores therein an operating system (OS), an application, and data (not shown) for realizing a function as a typical computer. In addition, the storage unit 407 stores the control program 409 therein. As described above, the input unit 110, the input unit 120, the CT reconstruction unit 130, the registration unit 140, the pixel value conversion unit 150, the CT reconstruction unit 160, and the output unit 170 illustrated in FIG. 1 can be realized by the control program 409.

The display unit 411 is a display apparatus for presenting the CT image generated by the CT reconstruction unit 160, for example. Specific examples of the display unit 411 include a liquid-crystal display and an organic EL (Electro-Luminescence) display. The input unit 413 is a device for receiving operation input. Specific examples of the input unit 413 can include a keyboard, a mouse, and touch panel.

The image processing apparatus 100 does not necessarily need to include the display unit 411 and the input unit 413. The display unit 411 and the input unit 413 may be connected to the image processing apparatus 100 from the outside via various interfaces such as an USB (Universal Serial Bus) or a display port.

(6. Effect According to this Embodiment)

The image processing apparatus 100 according to this embodiment generates the CT image by the IR method with use of the sinogram information and the prior CT image prepared in advance. Even when sufficient information content cannot be acquired only with the sinogram information, the CT image can be suitably generated by supplying the information with the information of the prior CT image. In particular, even when the field of view is not sufficient only with the sinogram information and the entire object cannot be restored, the area that can be suitably restored can be broadened with use of the prior CT image. As a result, the radiation amount that is actually applied in the therapy and the like can be calculated by restoring the image of the entire tomogram of the patient with use of an image having a narrow field of view photographed by the radiation therapy apparatus 200, for example.

(7. Notes)

The configuration of the abovementioned embodiment may be combined, or partial configuration portions thereof may be replaced. The configuration of the present invention is not limited to the abovementioned embodiment, and various modifications may be made without departing from the gist of the present invention. In particular, Expressions (1) to (4) are only examples, and other expressions may be applied.

### REFERENCE SIGNS LIST

1 Image processing system

100 Image processing apparatus

110 Input unit

120 Input unit

130 CT reconstruction unit

140 Registration unit

150 Pixel value conversion unit

160 CT reconstruction unit

170 Output unit

200 Radiation therapy apparatus

401 Control unit

403 RAM

405 Communication interface unit

407 Storage unit

409 Control program

411 Display unit

413 Input unit

415 Bus line

1. An image processing apparatus, comprising:
first input means for receiving input of sinogram information acquired by projecting radiation onto an object;
means for configuring a first tomographic image of the object from the sinogram information;
second input means for receiving input of a prior tomographic image obtained by imaging the object before the sinogram information;
conversion means for converting a pixel value of the prior tomographic image on the basis of a pixel value of the first tomographic image; and
means for generating a second tomographic image from the sinogram information with use of the prior tomographic image, the pixel value of which has been converted.

2. The image processing apparatus of claim 1, further comprising means for registering the first tomographic image and the prior tomographic image with each other, wherein the conversion means converts a pixel value of the prior tomographic image that has been registered.

3. The image processing apparatus of claim 1, wherein the second tomographic image is generated from the sinogram information by an iterative reconstruction method with use of the prior tomographic image that has been converted.

4. The image processing apparatus of claim 1, wherein the second tomographic image is generated from the sinogram information by a filtered-back projection method with use of the prior tomographic image that has been converted.

5. The image processing apparatus of claim 1, wherein the first tomographic image has a narrower field of view than the prior tomographic image.

6. The image processing apparatus of claim 1, wherein the sinogram information is imaged by a radiation therapy apparatus.

7. An image processing method performed by an image processing apparatus, which comprises:
the step of receiving input of sinogram information acquired by projecting radiation onto an object;
the step of configuring a first tomographic image of the object from the sinogram information;
the step of receiving input of a prior tomographic image obtained by imaging the object before the sinogram information;
the step of converting a pixel value of the prior tomographic image on the basis of a pixel value of the first tomographic image; and
the step of generating a second tomographic image from the sinogram information with use of the prior tomographic image, the pixel value of which has been converted.
